(19) United States
(12) Patent Application Publication
(10) Pub. No.: US 2001/0041813 A1
(43) Pub. Date: Nov. 15, 2001

(54) Process for the preparation of urea

(76) Inventors: Kees Jonckers, Susteren (NL); Jozef H. Meessen, Gulpen (NL)

Correspondence Address: PILLSBURY WINTHROP LLP, 1600 TYSONS BOULEVARD, MCLEAN, VA, 22102, US

(21) Appl. No.: 09/750,340

(22) Filed: Dec. 29, 2000

Related U.S. Application Data
(63) Continuation of application No. PCT/NL99/00396, filed on Jun. 29, 1999.

(30) Foreign Application Priority Data
Jun. 28, 1999 (NL) 1009516

(51) Int. Cl. C07C 273/04

(52) U.S. Cl. 564/67

(57) ABSTRACT

Process for the preparation of urea from ammonia and carbon dioxide in which a submerged condenser is used as high-pressure carbamate condenser and the urea synthesis solution leaving the submerged condenser is transferred to the reactor by means of an ejector. In particular, this relates to a process in which a pool condenser is used as submerged condenser.

The invention relates to a process for the preparation of urea from ammonia and carbon dioxide.

Urea can be prepared by reacting ammonia and carbon dioxide in a synthesis zone at a suitable pressure (for example 12-40 MPa) and temperature (for example 160-250° C.) to produce ammonium carbamate according to the reaction:

nNH₃+CO₂→H₂N—CO—ONH₄+(n-2)NH₃

and then dehydrating the resulting ammonium carbamate to produce urea according to the equilibrium reaction:

H₂N—CO—ONH₄→H₂N—CO—NH₂+H₂O

The degree to which these reactions proceed depends on, among other factors, the reaction temperature and pressure and the amount of excess ammonia present. The reaction product is a solution consisting mainly of urea, water, ammonia, and ammonium carbamate. To obtain the desired urea product, the ammonium carbamate and the ammonia must be removed from the reaction product, preferably for recycle into the synthesis zone. In addition to the reaction product solution, a gas mixture forms in the synthesis zone. This gas mixture comprises mainly ammonia and carbon dioxide, but may include minor amounts of nitrogen, oxygen, or other inert gases. It is preferable to remove the ammonia and carbon dioxide from the gas mixture for recycle into the synthesis zone. The referenced synthesis zone may, in practice, comprise a plurality of separate zones for forming ammonium carbamate and urea. These separate zones may be configured in separate pieces of apparatus or may, however, be combined in a single pressure vessel.

In practice, a variety of different methods have been used in commercial urea production plants. In the 1960's, urea was typically prepared in plants utilizing the so-called conventional high-pressure process. Toward the late 1960's, however, these conventional high-pressure plants began to be replaced by plants utilizing the so-called urea stripping process.

Urea plants utilizing the conventional high-pressure process are generally understood to be those plants in which the decomposition of the unconverted ammonium carbamate and the separation of the excess ammonia excess occurs at a pressure that is substantially lower pressure than the pressure in the synthesis reactor itself. In a conventional high-pressure urea plant the synthesis reactor is usually operated at a temperature of 180-250° C. and a pressure of 15-40 MPa with ammonia and carbon dioxide being fed directly into the synthesis reactor. In such conventional high-pressure processes, the molar ratio of the ammonia and carbon dioxide fed into the reactor (the N/C ratio) is typically maintained in range of 3 to 6.

In contrast, a urea stripping plant is understood to be one in which the majority of the unconverted ammonium carbamate is decomposed and the majority of the excess ammonia is removed at pressures nearly the same as the pressure in the synthesis reactor. This decomposition and removal occurs in one or more stripper(s) installed downstream of the synthesis reactor. Although thermal stripping may be used, more typically, the reaction product is fed into one or more strippers where a combination of heat and a stripping gas decompose the ammonium carbamate and remove the majority of the carbon dioxide and ammonia from the solution. The stripping gas is generally carbon dioxide, but ammonia, either singly or in combination with the carbon dioxide may also be used. The gas stream coming from the stripper comprises mainly ammonia and carbon dioxide and is typically fed into a high-pressure carbamate condenser to produce an ammonium carbamate solution that can be fed back into the synthesis reactor.

The mixture of unreacted gases that form in urea synthesis section is generally removed via a blow-down stream. In addition to the condensable ammonia and carbon dioxide, this gas mixture (reactor off-gas) may also contain inert gases such as nitrogen, oxygen, and possibly hydrogen. These inert gases may enter the reactor as minor components in the raw reaction gas feeds or as make-up air intended to provide corrosion protection. This gas mixture may be removed from the system immediately downstream of the reactor or downstream of the high-pressure carbamate condenser, depending on the process route chosen.

The condensable components (ammonia and carbon dioxide) can be absorbed, for example, in a high-pressure scrubber operating at or near the synthesis pressure before the inert gases are blown down. In such a high-pressure scrubber the condensable components, ammonia and carbon dioxide, are preferably absorbed from the reactor off-gas into a low-pressure carbamate stream. The carbamate stream from the high-pressure scrubber, with the absorbed ammonia and carbon dioxide, may then be returned to the synthesis reactor via the high-pressure carbamate condenser. It is also possible to incorporate a heat exchanger into the scrubber that can be utilized either singly or in combination with adsorption. The reactor, high-pressure scrubber, stripper, and high-pressure carbamate condenser are the most important components in the high-pressure section of a urea stripping plant.

In a urea stripping plant the synthesis reactor is typically operated at a temperature of 160-240° C., preferably at a temperature of 170-220° C., and at a pressure of 12-21 MPa, preferably 12.5-19 MPa. The steam consumption in a urea stripping plant is approximately 925 kg of steam per ton of urea. The N/C ratio in the synthesis in a stripping plant is generally maintained between 2.5 and 5. The synthesis can be carried out in one or two reactors. When using two reactors, the first reactor can be operated using only fresh raw material feeds and the second reactor can be operated either using only fresh raw material feeds or, more preferably, entirely or partly using recycle feed streams from the condenser or urea recovery units.

A frequently used configuration for urea stripping plants is referred to as the Stamicarbon CO₂stripping process and is described in European Chemical News, Urea Supplement, of Jan. 17, 1969, pages 17-20. In this process, which may incorporate one or more strippers, the urea synthesis solution from the reactor is stripped at or near the synthesis pressure by bringing the solution into countercurrent contact with gaseous carbon dioxide while heating the mixture. This stripping treatment decomposes the majority of the ammonium carbamate present into ammonia and carbon dioxide. The decomposition products and the additional carbon dioxide, along with a small amount of water vapor, are then removed from the solution in gaseous form and discharged. A majority of the gas mixture removed from the stripper is condensed and adsorbed in a high-pressure carbamate condenser, from which a high-pressure ammonium carbamate stream is returned to the synthesis reactor. The stripped urea synthesis solution is then fed into a urea recovery unit.

The high-pressure carbamate condenser is preferably configured as a so-called submerged condenser of the type described in NL-A-8400839. The gas mixture and a dilute carbamate solution from the high-pressure scrubber are introduced into the shell-side space of a shell-and-tube heat exchanger. A portion of the heat released by the resulting dissolution and condensation in the shell-side space is then removed by a medium flowing through tubes, for example water, to produce low-pressure steam. The submerged condenser can be oriented horizontally or vertically. It is, however, particularly advantageous to orient the submerged condenser horizontally (a so-called pool condenser; see, for example, Nitrogen, No. 222, July-August 1996, pp. 29-31) to provide relatively longer residence times for the liquid. In comparsion to other condenser designs, the longer residence time provided in a pool condenser increases the formation of urea. The increased quantity of urea raises the boiling point of the solution, allowing a greater temperature difference to be maintained between the solution and the cooling medium and increasing the efficiency of the heat transfer. The amount of urea formed in the pool condenser is typically at least 30% of the amount of urea that could theoretically be formed.

In the urea recovery unit, the pressure is reduced on the stripped urea synthesis solution and the majority of the remaining solvent is evaporated to recover the desired urea product. Depending on the amount of carbamate removed in the stripper, the urea recovery may be carried out in one or more pressure steps. The carbamate removed at reduced pressure in the urea recovery unit results in a low-pressure carbamate stream that is preferably recycled to the synthesis reactor via the high-pressure scrubber. In the high-pressure scrubber, this low-pressure carbamate stream is used to scrub non-converted ammonia and carbon dioxide from the gas mixture blown down from the synthesis section.

In the pool condenser, the gas stream from the stripper is condensed into the carbamate stream from the high-pressure scrubber. Since urea formation takes place in the pool condenser, a urea synthesis solution is obtained in the pool condenser. The urea synthesis solution leaving the pool condenser is transferred to the synthesis reactor together with the ammonia needed for the reaction. The synthesis reactor and the pool condenser are usually placed above the stripper in order to be able to make use of gravity in recycling the high-pressure stripper off-gases to the reactor.

With the present invention, it has been found that an improved process can be obtained by using a submerged condenser as the high-pressure carbamate condenser and transferring the urea synthesis solution from the submerged condenser to the synthesis reactor by means of an ejector. Preferably, a pool condenser is used as submerged condenser and the ammonia needed for the reaction is used to drive the ejector. The use of an ejector results in an extra head of 0.25 MPa, so that the pool condenser and the synthesis reactor can be installed at ground level. This not only is advantageous from the point of view of ease of operation and maintenance, but also involves lower investments in high-pressure, corrosion-resistant piping.

In a preferred embodiment of the present invention, both the gas stream leaving the stripper and the reactor off-gas are condensed in the submerged condenser with the resulting urea synthesis solution then being transferred from the submerged condenser to the reactor via an ejector. The use of a pool condenser as the submerged condenser is especially preferred with the ejector being preferably driven by the ammonia needed for the reaction. CO₂ gas strippers are preferred for stripping the urea synthesis solution leaving the reactor. The gas streams from the stripper and the reactor may be fed separately into the pool condenser or may be combined and fed into the pool condenser as a single stream. In this preferred embodiment, is it advantageous for a high-pressure scrubber to be installed in the blow-down stream leaving the pool condenser. This high-pressure scrubber preferably works as an adiabatic absorber or as a heat exchanger. Use of a combination of absorber and heat exchanger is also possible.

In other embodiments of the present invention, the functions of reactor, pool condenser and high-pressure scrubber may be combined in one or two high-pressure vessels, the functional portions of the vessel associated with these process steps being separated by low-pressure internals (designed for small pressure differences) with in these high-pressure vessels. By reducing the amount of high-pressure piping, these embodiments provide further practical advantages both by substantially reducing the capital investment associated with high-pressure piping and enhancing plant reliability by reducing the number of leakage sensitive high-pressure connections between piping and equipment is greatly reduced. Examples of these additional embodiments are:

combining a pool condenser with a horizontal reactor

integrating the scrubber into the pool condenser

integrating the scrubber into the reactor

combining the scrubber, pool condenser, and reactor into a single high-pressure vessel.

The invention is eminently suited for permitting equipment configurations and combinations that reduce the energy consumption. If, for example, use is made of a heat exchange between the off-gases from the first dissociation step following after the stripping treatment (so the off-gases from part of the dissociation processing unit) and the evaporation unit of the urea plant, it was, surprisingly, found that total steam consumption in the urea production drops to about 564 kg steam per ton of urea produced. This synergistic effect is made possible by the use of a pool condenser in combination with a high-pressure CO₂ stripper and an NH₃-driven ejector installed at a point in the process where at least 30% of the total amount of urea theoretically possible has been formed. Those skilled in the art with appreciate that there are more possibilities to exploit these synergistic effects in variants of the disclosed embodiments, for example passing a portion of the CO₂ feed to the reactor, or by optimizing the location and the design of the inert blow-down stream. These types of variations will be influenced by local preferences and conditions (greater ease of operation, lower investments, lower energy consumption), which will applied by one skilled in the art in the customary optimization process during the design phase of a project.

It has further been found that the present invention may be applied in improving and optimizing existing urea plants. Both conventional high-pressure urea plants and urea stripping plants can be debottlenecked with very good results by the addition of a submerged condenser, preferably a pool condenser, and an ejector.

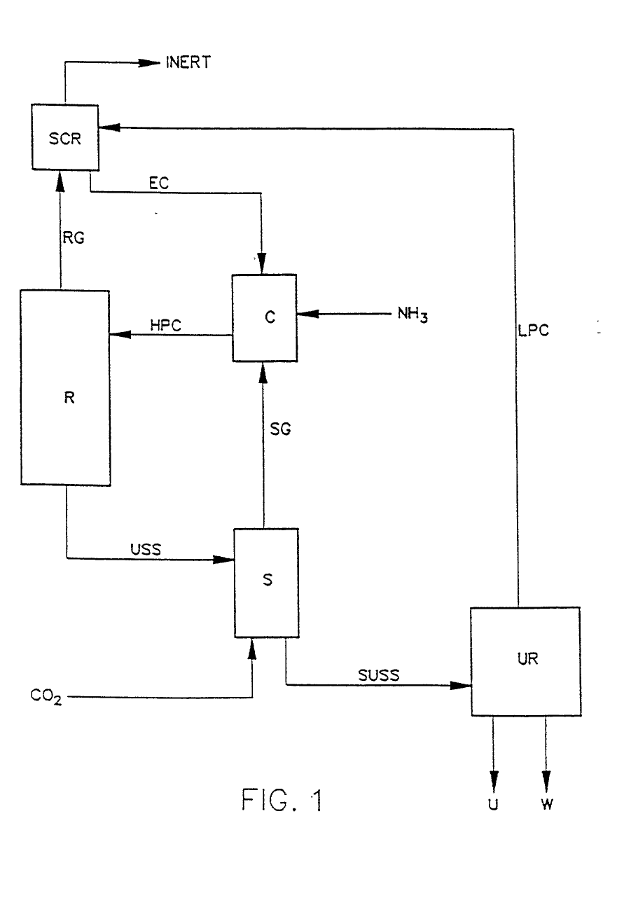

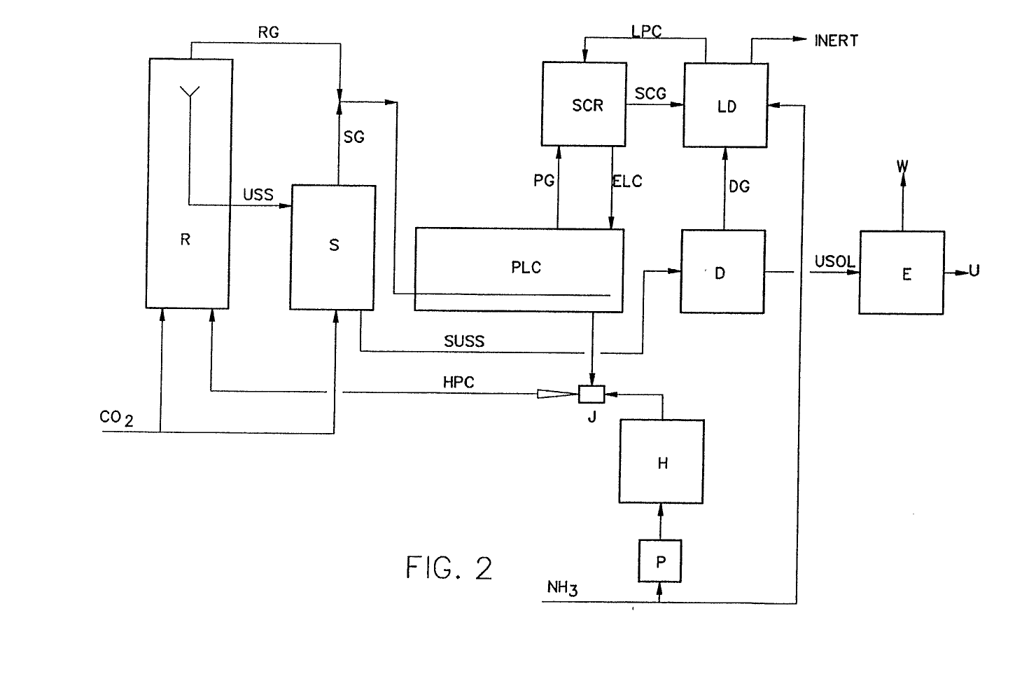

The present invention will be further described below with reference to FIGS. 1 and 2, with FIG. 1 representing the state of the art and FIG. 2 illustrating an embodiment of the present invention.

FIG. 1: A schematic diagram of part of a urea stripping plant according to the Stamicarbon CO₂ stripping process

FIG. 2: A schematic diagram of part of a urea stripping plant according to the Stamicarbon CO₂ stripping process modified according to the present invention by the addition of a pool condenser and an ejector.

In FIG. 1, R represents a reactor in a Stamicarbon CO₂ stripping plant in which carbon dioxide and ammonia are converted into urea. The urea synthesis solution (USS) leaving the reactor is transferred to a CO₂ stripper (S), where the USS is converted into a gas stream (SG) and a liquid stream (SUSS) by stripping with CO₂. The gas stream leaving the CO₂ stripper consists substantially of ammonia and carbon dioxide and the SUSS is the stripped USS. The stream containing the stripped urea synthesis solution SUSS is transferred to the urea recovery unit (UR), where the urea (U) is recovered and water (W) is discharged. In the UR, a low-pressure ammonium carbamate stream (LPC) is obtained, which is fed to the high-pressure scrubber (SCR). In this scrubber, the LPC is brought into contact with the gas stream coming from the reactor (RG), which consists substantially of ammonia and carbon dioxide, but which also contains the inert components (non-condensable components such as N₂, O₂, and perhaps H₂) present in the carbon dioxide and ammonia feed streams. The enriched carbamate stream (EC) coming from the SCR is transferred to the high-pressure carbamate condenser (C), in which the SG stream is condensed with the aid of EC. The resulting high-pressure carbamate stream (HPC) is then returned to the reactor. In this example, the fresh ammonia is shown as being fed only into the high-pressure carbamate condenser (C), but it can of course also be fed to a different point in the R→S→C→R loop or in the R→SCR→C→R loop.

FIG. 2 schematically represents a possible way of incorporating a pool condenser (PLC) and an extra ejector (J) in a Stamicarbon CO₂ stripping plant to obtain some of the advantages of the present invention. In FIG. 2, R represents a reactor in which carbon dioxide and ammonia are converted into urea. The urea synthesis solution (USS) leaving the reactor is passed to a CO₂ stripper (S), where the USS is converted into a gas stream (SG) and a liquid stream (SUSS) by stripping with CO₂. The gas stream (SG) leaving the CO₂ stripper consists substantially of ammonia and carbon dioxide and the SUSS is the stripped USS. The stream containing the stripped urea synthesis solution SUSS is transferred to the dissociation processing unit (D), where the SUSS is converted into a urea solution (USOL) and the gas mixture (DG) substantially consisting of ammonia and carbon dioxide from the dissociation. The USOL is transferred to the evaporation unit (E), where urea (U) is recovered and water (W) is discharged. The gas mixture DG is condensed in the low-pressure processing unit (LD). A low-pressure ammonium carbamate stream (LPC) is obtained from the LD, which is then fed to the scrubber (SCR). In the scrubber, the LPC is contacted with the gas stream (PG) from the pool condenser (PLC), which consists substantially of ammonia and carbon dioxide, but which also contains the inert components (non-condensable components) from the carbon dioxide and ammonia feed streams, fed to the PG with the reactor off-gas (RG) via the pool condenser. The enriched carbamate stream (ELC) coming from the SCR is returned to the pool condenser, in which the SG and RG streams are condensed with the aid of the ELC. The resulting urea synthesis solution, which already contains a substantial proportion of the urea formed in the pool condenser, is returned to the reactor via an ammonia-driven ejector (J). Fresh ammonia is supplied to the ejector (J) via pump (P) and heater (H). The SCG gas mixture leaving the scrubber, consisting substantially of inert gases and some ammonia and carbon dioxide, is condensed in LD, after which the inert gases are discharged from the system. To achieve an optimum N/C ratio in the tail end, ammonia or carbon dioxide can be fed to the LD as necessary. To reduce the plant energy consumption, the heat released during condensation in the pool condenser (PLC) can, for example, be used in the dissociation processing unit. Similarly, the heat released by condensation in the low-pressure processing unit (LD) can be used, for example, in the evaporation unit (E).

The benefits of the present invention will be further elucidated with reference to the following examples:

### EXAMPLE 1

In a urea plant as schematically depicted in FIG. 2, ammonia and carbon dioxide were converted into urea according to the process set out below. Of a CO₂ feed flow consisting of 46,060 kg CO₂, 230 kg water, 1468 kg nitrogen and 215 kg oxygen, 37,869 kg was transferred to the CO₂ stripper (S) and 8191 kg to the reactor (R). The temperature of this CO₂ feed was 120° C. and the pressure 14 MPa. The NH₃ feed stream, consisting of 35,609 kg NH₃ and 143 kg water, was split into two streams, of which the smaller one (1940 kg) was transferred to the low-pressure processing unit (LD), while 33,669 kg was sent to the ammonia heater (H). In this heater the NH₃ was heated from 40° C. to 135° C. and sent to the ejector (J) for use as driving gas. This ejector was fed with the urea synthesis solution from the pool condenser (PLC), consisting of 39,070 kg urea, 125 kg biuret, 53,815 kg NH₃, 54,419 kg CO₂ and 35,087 kg water, which was transferred from the ejector to the reactor with the aid of the NH₃ driving gas. This total stream (HPC) to the reactor had the following composition: 39,070 kg urea, 125 kg biuret, 87,484 kg NH₃, 54,419 kg CO₂ and 35,222 kg water. From this total stream, together with the small CO₂ feed stream to the reactor, urea was formed at a temperature of 183° C. and a pressure of 14 MPa. The resulting urea synthesis solution (USS) contained 69,465 kg urea, 222 kg biuret, 68,692 kg NH₃, 39,100 kg CO₂ and 44,302 kg water and was stripped in the CO₂ stripper (S) with the above-mentioned 37,869 kg CO₂. The temperature in the CO₂ stripper averaged 184° C. and the pressure was 14 MPa. The stripped urea synthesis solution (SUSS), with as composition 64,141 kg urea, 240 kg biuret, 15,012 kg NH₃, 17,636 kg CO₂, 37,972 kg water, 24 kg N₂ and 7 kg O₂, was transferred to the dissociation processing unit (D). In the dissociation processing unit (D) the stripped urea synthesis solution was split into a gaseous stream (DG) and a urea solution (USOL) consisting of 62,575 kg urea, 240 kg biuret and 19,227 kg water at a temperature of 135° C. and a pressure of 0.33 MPa. The gaseous stream (DG) contained 42 kg urea, 17,816 kg NH₃, 18,752 kg CO₂, 18,296 kg H₂O, 24 kg N₂ and 7 kg O₂ and was transferred to the low-pressure processing unit (LD), where it was converted, together with a small part of the NH₃ feed stream (1940 kg) and the gas stream (SCG) from the high-pressure scrubber, into the low-pressure carbamate stream (LPC). The urea solution leaving the dissociation processing unit (D) was transferred to the evaporation unit (E), where it was split into 62,575 kg urea (U), 240 kg biuret and 19,227 kg water (W). The evaporator temperature was 133° C. and its pressure 0.03 MPa. The reactor off-gas (RG) leaving the urea reactor had the following composition: 1505 kg NH₃, 1154 CO₂, 114 kg H₂O, 261 kg N₂ and 38 kg O₂. The gas from the CO₂ stripper (SG) consisted of 56,690 kg NH₃, 63,219 kg CO₂, 4927 kg H₂O, 1183 kg N₂ and 170 kg O₂. This stream was combined with the reactor off-gas (RG) and condensed in the pool condenser (PLC). The temperature in the pool condenser was 173° C. and the pressure 14 MPa. The urea synthesis solution leaving the pool condenser was transferred to the reactor via the ejector. The pool condenser off-gas (PG) consisted of 2979 kg NH₃, 10,455 kg CO₂, 239 kg H₂O, 1444 kg N₂ and 208 kg O₂ and was absorbed in the low-pressure carbamate stream (LPC) in the high-pressure scrubber. The low-pressure carbamate stream contained 42 kg urea, 18,046 kg NH₃, 22,690 kg CO₂ and 18,321 kg H₂O. From the high-pressure scrubber the gas stream (SCG) was transferred to the low-pressure processing unit (LD) and the high-pressure carbamate stream (ELC) was returned to the pool condenser. The gas stream (SCG) contained 229 kg NH₃, 3937 kg CO₂, 24 kg H₂O, 1444 kg N₂ and 208 kg O₂. From the low-pressure processing unit (LD), nitrogen and oxygen were blown down as inerts. The high-pressure carbamate stream (ELC) contained 42 kg urea, 20,795 kg NH₃, 29,207 kg CO₂ and 18,535 kg H₂O.

In this example the N/C ratio in the urea reactor was 3.1, the CO₂ conversion in the urea reactor 56.6%, and the CO₂ conversion in the pool condenser 34.4%. High-pressure steam consumption amounted to 910 kg steam per ton of urea produced.

### EXAMPLE 2

In a urea plant as schematically depicted in FIG. 2, ammonia and carbon dioxide were converted into urea according to the process set out below. Of a CO₂ feed stream consisting of 46,060 kg CO₂, 230 kg water, 1468 kg nitrogen and 215 kg oxygen, 37,849 kg was transferred to the CO₂ stripper (S) and 8210 kg to the reactor (R). The temperature of this CO₂ feed was 120° C. and the pressure 17.2 MPa. The NH₃ feed stream, consisting of 35,613 kg NH₃ and 143 kg water, was transferred to the ammonia heater (H). In this heater the NH₃ was heated from 40° C. to 135° C. and sent to the ejector (J) for use as driving gas. This ejector was fed with the urea synthesis solution from the pool condenser (PLC), consisting of 42,412 kg urea, 136 kg biuret, 56,257 kg NH₃, 35,128 kg CO₂ and 32,464 kg water, which was transferred from the ejector to the reactor with the aid of the NH₃ driving gas. This total stream (HPC) to the reactor had the following composition: 42,412 kg urea, 136 kg biuret, 91,869 kg NH₃, 35,128 kg CO₂ and 32,606 kg water. From this total stream, together with the small CO₂ feed stream to the reactor, urea was formed at a temperature of 191° C. and a pressure of 17.5 MPa. The resulting urea synthesis solution (USS) contained 67,160 kg urea, 215 kg biuret, 76,147 kg NH₃, 24,471 kg CO₂ and 39,930 kg water and was stripped in the CO₂ stripper (S) with the above-mentioned 37,849 kg CO₂. The temperature in the CO₂ stripper averaged 183° C. and the pressure was 17.2 MPa. The stripped urea synthesis solution (SUSS), with as composition 64,165 kg urea, 218 kg biuret, 19,906 kg NH₃, 22,010 kg CO₂, 32,267 kg water, 25 kg N₂ and 7 kg O₂, was transferred to the dissociation processing unit (D). In the dissociation processing unit (D) the stripped urea synthesis solution was split into a gaseous stream (DG) and a urea solution (USOL) consisting of 62,601 kg urea, 218 kg biuret and 19,227 kg water at a temperature of 155° C. and a pressure of 0.18 MPa. The gaseous stream (DG) contained 20,770 kg NH₃, 23,126 kg CO₂, 12,582 kg H₂O, 25 kg N₂, 7 kg O₂ and 41 kg urea and was transferred to the low-pressure processing unit (LD), where it was converted, together with the gas stream (SCG) from the high-pressure scrubber, into the low-pressure carbamate stream (LPC). In this example no ammonia was fed to the low-pressure processing unit (LD). The urea solution leaving the dissociation processing unit (D) was transferred to the evaporation unit (E), where it was split into 62,601 kg urea (U), 218 kg biuret and 19,227 kg water (W). The evaporation unit temperature was 133° C. and its pressure 0.03 MPa. The reactor off-gas (RG) leaving the urea reactor had the following composition: 1647 kg NH₃, 665 kg CO₂, 168 kg H₂O, 262 kg N₂ and 38 kg O₂. The gas from the CO₂ stripper (SG) consisted of 57,938 kg NH₃, 42,502 kg CO₂, 6955 kg H₂O, 1182 kg N₂ and 170 kg O₂. This stream was combined with the reactor off-gas (RG) and condensed in the pool condenser (PLC). The temperature in the pool condenser was 185° C. and the pressure 17.2 MPa. The urea synthesis solution leaving the pool condenser was transferred to the reactor via the ejector. The pool condenser off-gas (PG) consisted of 5422 kg NH₃, 3810 kg CO₂, 370 kg H₂O, 1443 kg N₂ and 208 kg O₂ and was absorbed in the low-pressure carbamate stream (LPC) in the high-pressure scrubber. The low-pressure carbamate stream contained 21,184 kg NH₃, 23,436 kg CO₂, 12,597 kg H₂O and 41 kg urea. From the high-pressure scrubber the gas stream (SCG) was transferred to the low-pressure processing unit (LD) and the high-pressure carbamate stream (ELC) was returned to the pool condenser. The gas stream (SCG) contained 413 kg NH₃, 309 kg CO₂, 13 kg H₂O, 1443 kg N₂ and 208 kg O₂. From the low-pressure processing unit (LD), nitrogen and oxygen were blown down as inerts. The high-pressure carbamate stream (ELC) contained 41 kg urea, 26,193 kg NH₃, 26,936 kg CO₂ and 12,953 kg H₂O. In this example the N/C ratio in the urea reactor was 4.0, the CO₂ conversion in the urea reactor was 66.8%, and the CO₂ conversion in the pool condenser was 47%. High-pressure steam consumption amounted to 564 kg steam per ton of urea produced.

1. Process for the preparation of urea from ammonia and carbon dioxide, characterized in that as high-pressure carbamate condenser use is made of a submerged condenser and the urea synthesis solution leaving the submerged condenser is transferred to the reactor by means of an ejector.

2. Process according to claim 1, characterized in that as submerged condenser use is made of a pool condenser.

3. Process according to claims 1-2, characterized in that the ejector is driven by the ammonia needed for the reaction.

4. Process according to any one of claims 1-3, characterized in that both the gas stream leaving the stripper and the reactor off-gas are condensed in the submerged condenser, following which the urea synthesis solution leaving the submerged condenser is transferred to the reactor via an ejector.

5. Process according to any one of claim 4, characterized in that as stripper use is made of a CO₂ stripper.

6. Process according to any one of claims 1-5, characterized in that a high-pressure scrubber is included in the blow-down stream leaving the pool condenser.

7. Process according to claim 6, characterized in that the high-pressure scrubber is an adiabatically operating absorber.

8. Process according to claim 6, characterized in that the high-pressure scrubber is a heat exchanger.

9. Process according to claim 6, characterized in that the high-pressure scrubber is a combination of absorber and heat exchanger.

10. Process according to any one of claims 1-8, characterized in that the functions of reactor, pool condenser and high-pressure scrubber are combined in one or two high-pressure vessels, the functionalities of these process steps being separated by means of low-pressure internals in these high-pressure vessels.

11. Method for improving and optimizing an existing urea plant, characterized in a submerged condenser for use as high-pressure carbamate condenser and an ejector for transferring the urea synthesis solution leaving the submerged condenser to the reactor are additionally installed.

14. Method according to claim 11, characterized in that as submerged condenser use is made of a pool condenser.
